(19) United States
(12) Patent Application Publication
(10) Pub. No.: US 2015/0216823 A1
(43) Pub. Date: Aug. 6, 2015

(54) COMPOSITIONS FOR THE TREATMENT OF MIGRAINE HEADACHES AND METHODS THEREOF

(71) Applicant: VR1, Inc., New York, NY (US)

(72) Inventor: Anjan Chatterjee, New York, NY (US)

(21) Appl. No.: 14/685,380

(22) Filed: Apr. 13, 2015

Related U.S. Application Data
(63) Continuation of application No. 13/974,624, filed on Aug. 23, 2013.
(60) Provisional application No. 61/692,826, filed on Aug. 24, 2012.

(51) Int. Cl. A61K 31/165; A61K 45/06; A61K 33/00

(52) U.S. Cl. CPC A61K 31/165; A61K 33/00; A61K 45/06

(57) ABSTRACT

Provided herein formulations of capsaicin for delivery by nasal route and methods of using the formulations for the treatment of migraines and other severe headaches. The formulations described herein contain mucoadhesives to optimize the therapeutic effect of capsaicin by intranasal delivery.

### CROSS REFERENCE TO RELATED APPLICATION(S)

This application is a continuation application of U.S. application Ser. No. 13/974,624 filed Aug. 23, 2013, now pending; which claims the benefit of priority under 35 U.S.C. §119(e) of U.S. Ser. No. 61/692,826, filed Aug. 24, 2012, the entire content of which is incorporated herein by reference.

### BACKGROUND OF THE INVENTION

1. Field of the Invention

The field of the invention is generally the treatment of migraine, cluster headaches and other severe headaches, and more specifically, a new nasal spray formulation for the treatment thereof.

2. Background Information

Headaches may be the most ubiquitous medical condition of mankind and it is estimated that one in three people will experience a severe headache at some stage in their life. Many different kinds of primary headache syndromes have been described, including migraine, cluster headache, medication overuse headache and most commonly tension type headache.

Migraine ranks in the top 20 of the world's most disabling medical illnesses. About 18% of all women and 6% of men suffer from migraine. In the United States alone, there are more than 33 million migraine sufferers. Of these, over 60% are women between 18 and 49 years of age. American employers lose more than $13 billion each year as a result of 113 million lost workdays attributable to migraines.

Cluster headache, originally named Horton's Cephalalgia or Horton's Headache after B. T Horton, who postulated the first theory as to their pathogenesis, is one of the most painful recurrent headaches that afflicts individuals. Some people affected with cluster headache have committed suicide, leading to the nickname “suicide headaches.” The headaches occur in “clusters” (periods of repeated attacks separated by symptom free intervals), and last up to 3 hours or more if untreated.

The cardinal symptoms of the cluster headache attack are the severe or very severe unilateral orbital, supraorbital and/or temporal pain, and the attack frequency of one to 16 attacks in 48 hours. The headache is accompanied by at least one of the following autonomic symptoms: ptosis (drooping eyelid), miosis (pupil constriction) conjunctival injection (redness of the conjunctiva), lacrimation (tearing), rhinorrhea (runny nose), and, less commonly, facial blushing, swelling, or sweating, all appearing on the same side of the head as the pain. The attack is also associated with restlessness, the sufferer often pacing the room or rocking back and forth. Less frequently, the sufferer will have an aversion to bright lights and loud noise during the attack. Nausea rarely accompanies a cluster headache, though it has been reported. The neck is often stiff or tender in the aftermath of a headache, with jaw or tooth pain sometimes present. Some sufferers report feeling as though their nose is clogged and that they are unable to breathe out of one of their nostrils.

The pain associated with cluster headaches is lancinating or boring/drilling in quality, and is located behind the eye (periorbital) or in the temple, sometimes radiating to the neck or shoulder. Analogies frequently used to describe the pain are a red-hot poker inserted into the eye, or a spike penetrating from the top of the head, behind one eye, radiating down to the neck, or sometimes having a leg amputated without any anesthetic. Patients are frequently incapacitated during the headaches and may be driven to suicidal thoughts and action during a cluster, reflecting the intensity of the discomfort.

Multiple options exist for the treatment of headache syndromes and span the range from non-pharmacological options to prescription drugs. Non-pharmacological options include relaxation techniques, biofeedback, yoga and stress reduction that may be helpful for tension headaches and as adjunctive therapy for migraine type headaches. Non-prescription medicines include over the counter (OTC) tablets and capsules such as TYLENOL®, BUFFERN®, MOTRIN® and ALLEVE®. Other non-prescription OTC alternatives commonly used in the United States include balms and lotions (for example, Tiger Balm) as well as other locally applied alternatives such as nasal capsaicin sprays (SINOL® and HEADACHE BUSTER®). Prescription medicines for migraine include the triptans (e.g., RELPAX® and MAXALT®) and dihydroergotamine (DHE). Non-specific prescription drugs for headache include opioids and other habit forming pain killers. In many cases patients may use one or more of these options in an effort to achieve acceptable pain control and frequently patients may use them simultaneously.

Currently available treatments, although very helpful, have several important limitations, as described below, that drive the medical need in this area in an effort to improve treatment.

Time to onset of effect: Most options require up to 45 to 60 minutes to start having a significant effect. Davies and Lipton (2000) reported on patient satisfaction with migraine therapy and concluded that satisfaction was negatively associated with severity of the headache and accompanying symptoms and positively associated with rapidity of onset of pain relief.

Insufficient efficacy: Frequently, one or more medicines need to be adjusted or changed for lack of or for insufficient efficacy. Increasing dosage and/or using multiple medications to improve pain relief is fraught with risk of adverse drug interaction and the risk of overdose (as with opioids) (see below).

Side effects: Non-steroidal anti-inflammatory (NSAIDs) analgesics (MOTRIN®, ALLEVE®) cause gastrointestinal irritation and ulcers. Between 10,000 and 20,000 patients die each year, from gastrointestinal bleeding associated with the use of NSAIDs. TYLENOL® has well documented liver toxicity associated with chronic use and the opioids are associated with respiratory depression and may be fatal in overdose.

Contraindications: Many current treatments have contraindications and/or warnings in special populations that limit their use. Triptans (e.g., MAXALT®, RELPAX®, IMITREX®) have restrictions in patients with a history of coronary artery disease and must be used “sparingly” because of their potential to cause coronary artery constriction.

Drug interactions: Drug interactions also limit the combined use of these medicines and potentially result in sub-optimal pain relief in many patients. Examples include triptans that may interact with several classes of antidepressants and must be used with caution in these patients and in conjunction with pain relievers such as ASPIRIN® and ibuprofen that may interact with blood thinners such as warfarin to increase the likelihood of bleeding.

Potential to cause dependence and/or medication overuse headache: Chronic use of opioids may cause physical dependence and many physicians under-dose these medications in an effort to reduce risk. OTC medications such as TYLENOL®EXCEDRIN®, ASPRIN and MOTRTN® are associated with medication overuse headache when used frequently and may compound the problem they are used to solve.

Medicaments may be systemically or locally delivered by intranasal administration, the nasal pathway offering a good route for systemic delivery of pharmaceuticals, such as hormones, for example, oxytocin and calcitonin, and analgesics, such as anti-migraine compositions, as the high blood flow and large surface area of the nasal tissues advantageously provides for rapid systemic uptake.

With respect to systemic delivery, studies have shown that significantly increased systemic bio-availability is achieved by delivery as a nasal spray as compared to drops. The systemic bio-availability of fluticasone propionate, where delivered both as an aqueous spray and as nasal drops, has been investigated, and the bio-availability of the nasal spray formulation was found to be about eight times higher than that of the nasal drop formulation. The intranasal administration of desmopressin as a nasal spray and as nasal drops has also been studied, and the bio-availability of the nasal spray formulation was two to three times higher than that of the nasal drop formulation. This has been attributed to an increased bio-availability of the nasal spray formulation to the by-passing of the nose and an increased absorption from the gastro-intestinal (GI) tract, where the nose is by-passed by one or both of mucociliary clearance of the substance or sniffing the substance along the floor of the nose and subsequent swallowing.

Using an intranasal or other mucosal route for systemic delivery of a therapeutic agent allows for ease of administration and the ability to bypass intestinal degradation and first pass hepatic metabolism of the therapeutic agent. There are times when it is desirable to not have systemic distribution of a therapeutic agent or to have a therapeutic agent targeted to a localized or regional area. For example, intranasal drug delivery has been used to bypass the blood-brain barrier and deliver substances to the central nervous system (CNS) and the brain.

Two nasal sprays, SINOL® and SINUS BUSTER®, containing capsaicin, are currently available over the counter in the United States. SINOL® and SINUS BUSTER®, but are targeted to patients with sinus headaches and seasonal allergic headaches (i.e., a broader population) and neither formulation is effective for treating or ameliorating symptoms associated with severe headaches or migraine/cluster headaches.

Capsaicin, when administered via nasal route, has an associated sting therefore SINOL® and SINUS BUSTER® have been intentionally formulated with low doses of capsaicin in order to be tolerable to a larger population of users, specifically those suffering from sinus and allergic headaches. SINOL® is 4× and SINUS BUSTER® is 4×/5×, which represent 0.001% and 0.001-0.0001% concentration of capsaicin, respectively.

In addition, these medicines contain feverfew (Pyrethrum parthenium) as additional active ingredients. Feverfew is contraindicated for use during pregnancy and lactation, rendering these compositions unsuitable for use specifically to treat headaches in a large portion of the population since the vast majority of migraine patients are women in their childbearing years.

A need therefore exists for a composition that has a rapid onset of action and can be used to safely and effectively treat migraines, frequent severe headaches and cluster headaches with fewer side effects and drug interactions than existing treatments.

### SUMMARY OF THE INVENTION

The present invention is based on the seminal discovery that a pharmaceutical composition containing capsaicin or Capsicum annuum 3×, formulated for nasal administration, is effective for the treatment of migraines and frequent severe headaches with a fast onset of action. This therapeutic effect may be enhanced by addition to the pharmaceutical composition of a mucoadhesive to improve binding and extend residence time on the nasal mucosa, maximizing the potential for absorption of capsaicin.

Provided herein are compositions formulated for nasal delivery comprising at least about 0.013% (w/w) capsaicin. The capsaicin in the compositions described herein may include at least one of Capsicum annuum, 3× Capsicum annuum, powdered capsaicin USP and/or oleoresin capsicum. In one embodiment, the composition further comprises an additional therapeutic agent. By way of example only, the composition may comprise ginger or zinger officinale as the additional therapeutic agent. In one embodiment, ginger is 3× ginger. In certain aspects, the compositions may further comprise a mucoadhesive agent including, but not limited to, microcrystalline cellulose. In other aspects, the compositions described herein are formulated for intranasal delivery by nasal spray.

In one embodiment, the concentration of capsaicin in the composition is from about 0.013% to 0.1% (w/w) capsaicin. In another embodiment, the concentration of capsaicin in the composition is from about 0.027% to 0.054% (w/w) capsaicin. In yet other embodiments, the concentration of capsaicin in the composition is about 0.0135% (w/w), 0.027% (w/w) or 0.054% (w/w) capsaicin. In a further embodiment, the concentration is from about 0.013% to 0.075% (w/w) capsaicin.

Also provided herein are methods for the treatment of a migraine, severe or cluster headache. The methods include administering to a subject in need thereof, a therapeutically effective amount of compositions formulated for nasal delivery comprising at least about 0.013% (w/w) of at least one of Capsicum annuum, 3× Capsicum annuum, powdered capsaicin USP and/or oleoresin capsicum, thereby treating the migraine or headache.

Provided herein are methods of inhibiting or preventing the symptoms of a migraine, severe or cluster headache by administering to a subject in need thereof, a therapeutically effective amount of compositions formulated for nasal delivery comprising at least about 0.013% (w/w) of at least one of Capsicum annuum, 3× Capsicum annuum, powdered capsaicin USP and/or oleoresin capsicum, thereby inhibiting or preventing the symptoms of the migraine or headache.

In certain aspects, the methods include administering compositions comprising at least about 0.013% (w/w) of at least one of Capsicum annuum, 3× Capsicum annuum, powdered capsaicin USP and/or oleoresin capsicum formulated for nasal delivery.

In certain aspects, the subject is a human. In one embodiment, the compositions described herein are administered by nasal spray with a suitable device. In other aspects, the methods described herein include further administering at least one additional therapeutic agent including, but not limited to, an analgesic. By way of example, analgesics to be administered in combination with the compositions described herein may include non-steroidal anti-inflammatory (NSAID) agents, triptans, opioids, acetaminophen, semi-synthetic opioids or dihydroergotamine (DHE). The additional therapeutic agent(s) may be administered simultaneously or in succession, after minutes, hours, or days, with the compositions described herein. In one aspect, the methods described herein further include administering oxygen to the subject in combination with the composition formulated for nasal delivery comprising at least about 0.013% (w/w) capsaicin.

In certain aspects, the compositions consist essentially of at least one of Capsicum annuum, 3× Capsicum annuum, powdered capsaicin USP and/or oleoresin capsicum. In other aspects, the compositions consist essentially of at least one of Capsicum annuum, 3× Capsicum annuum, powdered capsaicin USP and/or oleoresin capsicum and ginger.

In one embodiment, the compositions described herein are formulated in metered dose spray. In certain aspects, the frequency of administration is minutes, hours, days, weeks, or months.

### DETAILED DESCRIPTION OF THE INVENTION

Capsaicin, which is derived from chile peppers and is the cause of the “heat” from the pepper, has been known to have pain relieving properties for hundreds of years and has been used to topically treat pain on the body and over the joints. Capsaicin is safe for human ingestion and has been ingested in the form of chile peppers for millennia. As described herein, capsaicin has also been found to be effective in the treatment of migraine, severe headaches and cluster headaches.

There are two homeopathically active ingredients in the compositions and formulations of the disclosure: Capsicum annuum 3× (homeopathic active) for relief of pain associated with headaches and migraine and ginger 3× (homeopathic active) for the nausea and vomiting associated with severe headaches and migraine.

Capsaicin, which has antimicrobial and antifungal properties, also serves as a natural preservative. The broad antifungal and antimicrobial properties of capsaicin (both in the form of chile pepper extracts and capsaicin itself) have been studied in vitro in varied medical and agricultural/food literature and is summarized in Table 1 below. The antifungal literature is more detailed and suggests that capsaicin/chile peppers' antifungal effects may be related at least in part to the presence of a class of lipid transfer proteins (LTPs) and their associated inhibition of alpha amylase and L-trypsin (Ribeiro, S. F., et al., Antonie Van Leeuwenhoek, 2012, 101(3): p. 657-70).

Grapefruit seed extract (GSE) is also commonly used as a preservative (Cvetnic, Z. and S. Vladimir-Knezevic, Acta Pharm, 2004, 54(3): p. 243-50). Table 2 below outlines the data available on the preservative properties of GSE.

TABLE 1
Antimicrobial activity of Capsicum annuum (Capsaicin)

| Num- | | | | |
|---|---|---|---|---|
| ber | Moiety | Organism(s) | Class | Notes |
| 1 | Capsicum | S. Aureus, coliform, yeast | Bacteria | |
| | annuum | | and | |
| | | | fungi | |
| 2 | Capsicum | Candida Albicans, K | Fungi | CaT1 |
| | annuum | Marxiannuus, | | Peptide |
| | | S. Cerevisae | | fraction |
| 3 | Capsicum | Colletotrichum | Fungi | LTP |
| | annuum | lindemunthianum, Candida | | |
| | | Tropicalis | | |
| 4 | Capsaicin | Botrytis Cinerea, Aspergillus | Fungi | |
| | | Niger | | |
| 5 | Capsicum | Candida Albicans, | Fungi | Lipid |
| | annuum | Saccharomyces Cerevisae, | | transfer |
| | | Schizosaccharomyces pombe | | protein |
| | | | | (LTP) |
| | | | | fraction |
| 6 | Capsicum | K. Marxiannus, C. Tropicalis, | Fungi | 2S |
| | annum | C. Albicans, S. Cerevisiae | | Albumin |
| | | | | fraction |
| 7 | Capsaicin | Saccharomyces cerevisiae | Fungi | |
| 8 | Capsicum | Saccharomyces cerevisiae, | Fungi | AMP |
| | annuum | Candida Albicans, Candida | | |
| | | parapsilosis, Candida tropicalis, | | |
| | | Pichia membranifaciens, | | |
| | | kluyveromyces marxiannuus, | | |
| | | Candida guilliermondii | | |
| 9 | Capsicum | Candida albicans | Fungi | |
| | annuum | | | |
| 10 | Capsicum | Candida Spp, Aspergillus | Fungi | Cay-1 |
| | frutescens | fumigatus, Cryptococcus | | saponin |
| | | neoformans | | fraction |
| 11 | Capsaicin | E. Coli, B Subtilis, P. | Bacteria | |
| | | Solancearum | | |
| 12 | Capsaicin | S. Aureus | Bacteria | |
| 13 | Capsicum | Bacillus cereus, B. Subtilis, | Bacteria | |
| | annuum/ | Clostridium Sporogenes, | | |
| | Capsaicin | Clostridium tetani, | | |
| | | Streptococcus pyogenes | | |
| 14 | Capsicum | Staphylococcus species, E. Coli, | Bacteria | |
| | annuum | Bacillus aureus, B. Subtilis | | |
| 15 | Capsaicin | H Pylori | Bacteria | |
| 16 | Capsicum | Bacillus cereus, Bacillus | Bacteria | |
| | annuum | subtilis, Sarcina lutea, | | |
| | | Staphylococcus aureus | | |

TABLE 2
Antimicrobial activity of grapefruit seed extract (GSE)

| Num- | | | | |
|---|---|---|---|---|
| ber | Moeity | Organism(s) | Class | Notes |
| 1 | Grapefruit | Salmonella enteridis, | Bacteria | Ethanolic extract |
| | seed extract | Listeria | | |
| | | monocytogenes, | | |
| | | streptococcus | | |
| | | fecalis, Bacillus | | |
| | | subtilis and | | |
| | | other gram | | |
| | | positive bacteria | | |
| 2 | Grapefruit | Salmonella, Penicillium | Bacteria | Indirect based on |
| | seed extract | | and fungi | Benzethonium |
| | | | | chloride extracted |
| | | | | from GSE |

Compositions and formulations containing capsaicin USP, grapefruit seed extract and benzalkonium chloride as a preservative system have been developed for the compositions and formulations described herein. This preservative system has been shown to have surprisingly superior preservative and bactericidal effect.

New compositions and formulations containing capsaicin as an active ingredient have been developed specifically for the homeopathic treatment of migraine, cluster headaches and other severe headaches. The compositions and formulations disclosed herein are administered by intranasal delivery and provide rapid relief of the symptoms associated with migraines, cluster headaches and other severe headaches, as well as fewer side effects and drug interactions than existing treatments.

The tonicity of the formulations of the disclosure have been adjusted, and substances have been incorporated (e.g., aloe and glycerol), in order to increase tolerability of the sting upon administration without negatively impacting the efficacy of capsaicin.

Nasal delivery is expected to be advantageous for the administration of medicaments requiring a rapid onset of action, for example, analgesics, anti-emetics, insulin, anti-epileptics, sedatives and hypnotica, and also other pharmaceuticals, such as, cardio-vascular drugs.

It is envisaged that nasal administration will provide for a fast onset of action, at a rate similar to that of injection and at a rate much faster than that of oral administration. Indeed, for the treatment of many acute conditions, nasal administration is advantageous over oral administration, since gastric stasis can further slow the onset of action following oral administration.

Nasal administration may also be neural in nature and have an even faster onset of action than systemic delivery. When nasal administration leads to delivery of the analgesic to the central nervous system (CNS), desensitization of the local branch of V2 and depletion of neurotransmitters such as CGRP (calcitonin gene related peptide, which has been implicated in the genesis of a migraine or vascular headache by causing the dilation of intracranial blood vessels and local inflammation) may occur. Accordingly, pain relief from migraines and cluster headaches is afforded in a matter of seconds by inhibition of nerve transmission through the trigeminal nerve entering the brain. The speed of transmission or occurrence of the analgesic effect is substantially faster when it is neural in nature than if systemic in nature. In addition, a smaller amount analgesic is required to initiate and maintain analgesic effect resulting in nearly undetectable amounts of the drug in the systemic circulation. Administration of smaller amounts of analgesic reduces the risk of drug-drug interaction, issues with metabolism or drug excretion (i.e., hepatic or renal toxicity) and systemic side effects, and therefore allows for multiple dosing.

While not wanting to be bound by a particular theory, it is believed that the action of the compositions and formulations described herein are delivered to the CNS and cause desensitization of the local branch of V2, one of the three branches of the trigeminal or fifth cranial nerve. The trigeminal nerve is predominantly a sensory nerve and is the main sensory supply of the inside of the nose. In addition, V2 also supplies the intracranial blood vessels that course through the Dura mater. These blood vessels dilate during a migraine or vascular headache, resulting in stimulation of stretch receptors that result in the sensation of pain, transmitted to the brain by the aforementioned trigeminal nerve. Desensitization of the V2 branch by the application of capsaicin inside the nose, depletes neurotransmitters such as CGRP (calcitonin gene related peptide) that have been implicated in the pathology of migraine, cluster headaches and other severe headaches by causing the dilation of the blood vessels and inflammation locally. Together, these actions abort the migraine/headache by inhibiting the nerve transmission through the trigeminal nerve going to the brain (where we actually feel the pain). This cross inhibition is likely taking place in the trigeminal ganglion which expresses CGRP receptors and has been shown to be affected by application of capsaicin peripherally.

In addition, the pH of the formulations described herein have been developed to mirror the optimal pH at which the VR1 receptor “opens” thereby improving the action of capsaicin at this receptor. To this end, the pH of the formulations and compositions of the disclosure have been adjusted to a pH of between about 6 and 6.5.

The compositions and formulations disclosed herein are in the form of a nasal spray, which is absorbed rapidly and provides subjects suffering from migraines and severe headaches with relief within a minute or less. The compositions and formulations of the present disclosure are not absorbed into the body (i.e., remain local in the nose) and with the exception of a strong sensation/sting in the nose on application, will not have any systemic adverse effects or side effects. In addition, the compositions and formulations of the disclosure will not interact with other medications the patient may be taking and are non-habit forming.

The compositions and formulations disclosed herein address the specific needs of patients with migraine and severe headaches by providing rapid pain relief in contrast to other pain relief medications such as TYLENOL®, ADVIL® and EXCEDRITN®, which take up to an hour or more for relief.

The compositions containing capsaicin have been formulated to specifically address the pain relief needs of patients with migraine and severe headaches as opposed to other existing nasal sprays that are not formulated for, or effective at, treating severe pain.

The compositions of the disclosure are formulated for intranasal delivery and are not absorbed into the body, resulting in little if any drug interactions. Therefore, the compositions and formulations disclosed herein can be used in combination or in conjunction with other therapies and do not interact with other migraine or headache medicines.

As the compositions of the disclosure are formulated for nasal delivery, they does not cause stomach or liver problems like other conventional analgesics such as ADVIL®, EXCEDRIN®, and TYLENOL®. Additionally, the compositions and formulations are safe for patients with cardiovascular disease unlike RELPAX® or IMITREX® and are suitable for patients with kidney disease as they are not metabolized in the body.

The compositions of the disclosure, which contain the naturally derived active component, capsaicin, are formulated for administration as a homeopathic nasal spray and are not habit forming and therefore may be used as needed for the treatment of migraines, severe frequent headaches and cluster headaches unlike opioids for example.

Oleoresin capsicum (an oily plant extract containing a cocktail of capsaicin type compounds), powdered capsaicin USP, Capsicum annuum, and Capsicum annuum 3× were examined for use in the compositions and nasal spray formulations of the disclosure (see EXAMPLES section).

The ingredients described herein are formulated and mixed in accordance with the Homeopathic Pharmacopeia of the United States (HPUS) principles to ensure compliance with homeopathic regulation. In addition to Capsicum annuum, 3× Capsicum annuum, powdered capsaicin USP and/or oleoresin capsicum and ginger, the compositions and formulations contain inactive excipients as described in detail below.

The inactive ingredients of the disclosure (collectively referred to as “excipients”) are formulated specifically to improve patient compliance and maximize tolerability and efficacy.

An appropriate buffer, citrate buffer, by way of example only, (e.g., citric acid and sodium citrate), is added to the formulation to titrate the formulation pH to between about 6.0 and 6.5. This pH is closest to the natural pH of the nose (6.5) and is judged optimal for the function of the formulations of the disclosure and its tolerability by the patient. A pH of about 6.5 is thought to be ideal for the agonist action of capsaicin on the TRPV1 receptor (VR1 receptor), which subsequently initiates the cascade of neural events described above.

Sodium chloride (NaCl) is added on an as needed basis to maintain as isoosmotic a solution as possible for maximal tolerability by the patient.

Eucalyptol, glycerol and aloe are added to the formulation to soothe the nasal mucosa and improve tolerability of the spray.

AVICEL® RC 591 (microcrystalline cellulose) is added to the formulation as a mucoadhesive to improve binding on the nasal mucosa for optimization of the therapeutic effect.

Tween 80 is a surfactant used to assist in the creation of a homogenous (non-precipitating) solution by dissolution of water insoluble compounds.

Benzalkonium Chloride (BKC) in addition to the two natural preservatives discussed above, capsaicin and grapefruit seed extract (GSE), are included in the formulation.

The addition of a mucoadhesive agent in order to promote more efficient drug uptake by maximizing residence time of the capsaicin in the nasal mucosa was contemplated. Use of mucoadhesive agents in nasal drug delivery systems has been reported however, in general, mucoadhesive agents are relegated to powder or gel formulations rather than liquid formulations. Furthermore, a review of the composition of some commercially available liquid nasal spray products revealed that none of those investigated contain a mucoadhesive agent (e.g., DEMOSPRAY®, demopressin acetate; OCTIM®, demopressin acetate; RHINOLAST®, azelastine hydrochloride; IMIGRAN®, sumatriptan). Where mucoadhesives are conventionally used, they are in powder-form nasally administered products, in order to help keep the powder particles in contact with the nasal mucosa. The fact that mucoadhesives are not typically used in formulations may possibly be due to the fact that a mucoadhesive agent may cause blockage of the spray nozzle during patient use. Notwithstanding, a mucoadhesive agent was incorporated in the formulation described herein in order to extend the residence time of the capsaicin in the nasal mucosa thereby maximizing the potential for drug absorption and optimizing efficacy.

In one embodiment, the invention provides a composition suitable for nasal delivery comprising at least about 0.013% capsaicin. In one aspect, the concentration is from about 0.013%-0.1% capsaicin. In another aspect, the concentration is from about 0.027 to 0.054% capsaicin. In one aspect, the composition is formulated in a metered dose spray.

In one embodiment, the invention provides a method for the treatment of, inhibition of symptoms of or prevention of a migraine, severe or cluster headache comprising administering to a subject in need thereof, a therapeutically effective amount of the capsaicin composition described herein. Preferably, the subject is a human.

For the purposes of nasal administration, the compositions of the invention will preferably be in a container or device provided with means enabling application of the contained composition to the nasal mucosa, e.g., in a nasal applicator device. Suitable applicators are known in the art and include those adapted for administration of liquid compositions to the nasal mucosa in drop or spray form. Suitable administrators include, but are not limited to, e.g., atomizing devices, pump-atomizers and aerosol dispensers. In the latter case, the applicator can contain a composition in accordance with the invention together with a propellant medium suitable for use in a nasal applicator. The atomizing device will be provided with an appropriate spray adaptor allowing delivery of the contained composition to the nasal mucosa. Such devices are well known in the art.

The container, e.g., nasal applicator, may contain sufficient composition for a single nasal dosing or for the supply of several sequential dosages, e.g., over a period of minutes, hours, days or weeks. Quantities of individual dosages supplied will be in accordance with a physician's advice. The stability of the compositions of the invention may be determined in conventional manner.

When ranges of values are disclosed then, unless otherwise specified, this notation is intended to include the numbers themselves and the range between them. This range may be integral or continuous between and including the end values. By way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

The term “capsaicin” as used herein is intended to describe “Capsicum annuum,” “3× Capsicum annuum,” “powdered capsaicin USP,” and/or “oleoresin capsicum”, each of which independently refer to a form of “capsaicin” as a compound in the compositions and formulations described herein.

The term “combination therapy” means the administration of two or more therapeutic agents to treat a therapeutic condition or disorder described in the present disclosure. Such administration encompasses co-administration of these therapeutic agents in a substantially simultaneous manner, such as in a single intranasal dose having a fixed ratio of active ingredients or in multiple, separate doses for each active ingredient (e.g., intranasal doses or intranasal dose(s) and capsule(s)). In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The compound(s) of the present invention can exist as therapeutically acceptable salts. The present invention includes compound(s) listed above in the form of salts, including basic addition salts. Suitable salts include those formed with both organic and inorganic bases. Such base addition salts will normally be pharmaceutically acceptable. However, salts of non-pharmaceutically acceptable salts may be of utility in the preparation and purification of the compound in question. Acidic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. Basic addition salts can be prepared during the final isolation and purification of the compound(s) by reacting a phenoxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds of the present invention, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Any of the well-known techniques, carriers, and excipients may be used as suitable and as understood in the art; e.g., in Remington's Pharmaceutical Sciences. The pharmaceutical compositions disclosed herein may be manufactured in any manner known in the art.

The formulations include those suitable for nasal administration. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both.

Preferred unit dosage formulations are those containing an effective dose, as herein below recited, or an appropriate fraction thereof, of the active ingredient.

In certain instances, it may be appropriate to administer at least one of the compounds described herein (or a pharmaceutically acceptable salt, ester, or prodrug thereof) in combination with another therapeutic agent. By way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either by nasal delivery or by nasal delivery and as a pill). One of the therapeutic agents may be given in multiple doses, or both may be given as multiple doses. If not simultaneous, the timing between the multiple doses may be any duration of time ranging from a few minutes to four weeks.

The following examples are intended to illustrate but not limit the invention.

### Example 1

### Preparation of Stock Solution for Capsaicin Formulations

Examples 1-3 illustrate the development of capsaicin formulations without a mucoadhesive for administration as a nasal spray. This example demonstrates the preparation of a stock solution for capsaicin formulation experiments.

Capsaicin powder (95%) USP, sample obtained from Chillies Export House, India.

Polysorbate 80 (Tween 80) USP/Ph Eur, obtained from Sigma-Aldrich, UK.

Rosemary extract, Lot no 905113. 30 g sample obtained from Azelis, UK.

Eucalyptol, Lot no ECH B1/10556. 10 g sample obtained from Mane, UK.

Grapefruit seed extract (Citrus grandis, 10% dry hydroalcoholic extract), Lot No 1100512. 30 g sample obtained from EPO srl, Italy.

Glycerol USP/Ph Eur, Lot No K42075193. 1 kg sample obtained from Merck, Germany.

Ascorbic acid Ph Eur, Lot No 222U1. 50 g sample obtained from Mistral Chemicals, Northern Ireland.

Citric acid anhydrous USP/Ph Eur, Lot No K93210941. 5 kg sample obtained from Merck, Germany.

Sea salt, Lot No MM1177. 1.5 kg sample obtained from All in All Food Ingredients, Ireland.

Purified water, provided from laboratory purification system.

Nasal spray bottles (10 mL), provided as samples by MVW Healthcare.

Nasal spray pumps, snap-on (50 μL spray volume), provided as samples by MWV Healthcare.

Samples were to be manufactured as laboratory samples (i.e., not according to GMP specifications) and no product testing was performed.

Samples of capsaicin solution of 0.054, 0.027, 0.0135 and 0.0054% w/v were prepared. The samples were added to 10 mL bottles fitted with nasal spray pumps (to deliver a 50 μL per dose).

In order to dissolve the oleoresin capsicum in the aqueous vehicle, a small quantity of surfactant (e.g., 0.5% Tween 80) was required in the formulation. However, this addition led to the generation of a slightly turbid, red colored liquid, which was not aesthetically pleasing and could potentially present issues with patients (i.e., the possibility of red-colored droplets falling from the nose of a patient, which could stain clothing and/or cause alarm, if the patient were to believe they were suffering a nose bleed).

Therefore, it was decided to evaluate the use of the concentrated capsaicin (a white powder) as a replacement for the oily oleoresin capsicum extract. However, as capsaicin powder is hydrophobic it was necessary to determine what quantity of surfactant was necessary to maintain the capsaicin in solution. A study was therefore conducted to determine the required concentration of a chosen surfactant (Tween 80) to achieve and maintain complete dissolution of the capsaicin (assuming a capsaicin concentration of 0.054% w/v). In other words, the level of surfactant required for dissolving the capsaicin in an aqueous vehicle was determined as described below.

Determination of required concentration of Tween 80: A stock solution (not including capsaicin or Tween 80) was prepared according to the composition given below in Table 3.

TABLE 3
Composition of stock solution.

| Material | Quantity (g) |
|---|---|
| Rosemary extract | 0.40 |
| Eucalyptol | 0.65 |
| Grapefruit seed extract | 0.25 |
| Glycerol | 18.25 |
| Sea salt | 2.65 |
| Ascorbic acid | 4.15 |
| Citric acid | 1.30 |
| Purified water | To 500 mL |

The stock solution was prepared as follows: Sea salt, ascorbic acid, grapefruit seed extract and glycerol weighed into a 500 mL volumetric flask. Purified water (200 mL) added to the flask and contents mixed by swirling the flask. (Note: at this stage it was observed that the grapefruit seed extract imparts a noticeable straw color). Rosemary extract and eucalyptol added to the flask and flask swirled to mix contents (Note: rosemary extract also imparts noticeable coloration to the solution). Solution made up to volume (500 mL) by addition of purified water. At this stage the solution was very slightly turbid.

### Example 2

### Capsaicin Solubilization Experiments

This example illustrates concentration of surfactant required to achieve complete dissolution of the capsaicin of samples containing a range of capsaicin concentrations (0.054, 0.027, 0.0135 and 0.0054% w/v).

A series of experiments were undertaken to prepare various samples with the addition of a set quantity of capsaicin powder (equivalent to 0.05% in solution) and varying concentrations of Tween 80.

Sample 1: 1% Tween 80 was prepared according to the following protocol: 0.5 g; Tween 80; 0.025 g capsaicin; and 10 mL stock solution. The sample was sonicated for 2 minutes and then visually assessed. The capsaicin had completely dissolved. Solution made up to 50 mL with stock solution and sonicated for 2 minutes. Clear, light straw-colored solution obtained.

Sample 2: 0.5% Tween 80 was prepared according to the following protocol: 0.1 g Tween 80; 0.011 g capsaicin; and 10 mL stock solution. The sample was sonicated for 2 minutes and then visually assessed. The capsaicin had completely dissolved. Solution made up to 20 mL with stock solution and sonicated for 2 minutes. Clear, light straw-colored solution obtained.

Sample 3: 0.25% Tween 80 was prepared according to the following protocol: 0.05 g Tween 80; 0.011 g capsaicin; and 10 mL stock solution. The sample was sonicated for 2 minutes and then visually assessed. A slightly turbid solution resulted. Solution made up to 20 mL with stock solution and sonicated for 2 minutes. Very slightly hazy, light straw-colored solution obtained.

Sample 4: 0% Tween 80 was prepared according to the following protocol: 0.011 g capsaicin; and 10 mL stock solution. The sample was sonicated for 2 minutes and then visually assessed. A slightly turbid solution resulted. Solution made up to 20 mL with stock solution and sonicated for 2 minutes. Slightly hazy, light straw-colored solution obtained.

All samples were left overnight and visually assessed again on the following day. From the initial visual assessment it was considered that all samples containing Tween 80 (0.25% to 1%) were transparent solutions (i.e., capsaicin fully dissolved). However, from a closer examination of the Sample 3 (0.25% Tween 80) very fine particles in suspension were observed, giving the sample a slight haziness. All samples were re-examined after storage at room temperature for one week, however, no change in the appearance of the samples was observed. Based on the experimental data, it was concluded that the required concentration of Tween 80 in the stock solution to achieve complete dissolution of capsaicin (at 0.05-0.055% concentration) was 0.5% w/v.

### Example 3

### Manufacture of Product Samples without a Mucoadhesive

Samples of the formulation containing four strengths of capsaicin (0.054%, 0.027%, 0.0135% and 0.0054% w/v) were prepared. The samples were added to 10 mL nasal spray bottles fitted with a snap-on spray pump in order to deliver 50 μL dose.

A capsaicin (0.054% w/v) stock solution was prepared for use in preparation of varying strengths of capsaicin. A stock solution of the formulation was prepared as detailed in Table 4.

TABLE 4
Stock solution for capsaicin formulations without a mucoadhesive.

| Material | % w/v | Quantity (g) |
|---|---|---|
| Capsaicin powder | 0.054 | 0.27 |
| Rosemary extract | 0.08 | 0.40 |
| Eucalyptol | 0.13 | 0.65 |
| Grapefruit seed extract | 0.05 | 0.25 |
| Glycerol | 3.65 | 18.25 |
| Sea salt | 0.53 | 2.65 |
| Ascorbic acid | 0.83 | 4.15 |
| Citric acid | 0.26 | 1.30 |
| Tween 80 | 0.50 | 2.50 |
| Purified water | To 100 | To 500 mL |

The formulation was prepared by addition of each of the components to approximately 200 mL purified water in a 500 mL volumetric flask. The flask was agitated to dissolve the components in the water and then made up to 500 mL with purified water.

Capsaicin nasal spray samples were generated according to the following procedures.

0.054% w/v capsaicin (batch no. 0953/17/A): Aliquots of the stock solution (10 mL) were filled into 10 mL volume nasal spray bottles using a 10 mL syringe. Each bottle was then fitted with a spray pump to afford 20 bottles.

0.027% w/v capsaicin (batch no. 0953/17/B): Stock solution from Table 3 (250 mL) was added to a 500 mL volumetric flask and made up to volume with purified water. Aliquots of the resulting solution (10 mL) were filled into 10 mL volume nasal spray bottles using a 10 mL syringe. Each bottle was then fitted with a spray pump to afford 20 bottles.

0.0135% w/v capsaicin (batch no. 0953/17/C): Solution from batch no. 0953/17/B (250 mL) (i.e., 0.027% w/v capsaicin solution) was added to a 500 mL volumetric flask and made up to volume with purified water. Aliquots of the resulting solution (10 mL) were filled into 10 mL volume nasal spray bottles using a 10 mL syringe. Each bottle was then fitted with a spray pump to afford 20 bottles.

0.0054% w/v capsaicin (batch no. 0953/17/D): Solution from batch no. 0953/17/D (100 mL) (i.e., 0.0135% w/v capsaicin solution) was added to a 250 mL volumetric flask and made up to volume with purified water. Aliquots of the resulting solution (10 mL) were filled into 10 mL volume nasal spray bottles using a 10 mL syringe. Each bottle was then fitted with a spray pump to afford 20 bottles.

### Example 4

### Materials and Methods for Capsaicin Formulations Containing a Mucoadhesive

Examples 4-6 illustrate the development of additional capsaicin formulations without a mucoadhesive for administration as a nasal spray.

Preparation of a 50 mg/mL citrate buffer, pH 6: Using an analytical balance, an amount of 2.78 g of monohydrate citric acid and 3.89 g of dihydrate sodium citrate were weighed into individual 50 mL volumetric flasks and dissolved in water. The two resultant solutions were titrated together to produce a solution of pH 6 citrate buffer.

Preparation of a 100 mg/mL citrate buffer, pH 6: Using an analytical balance, an amount of 11.1 g of monohydrate citric acid and 15.6 g of dihydrate sodium citrate were weighed into individual 100 mL volumetric flasks and dissolved in water. The two resultant solutions were titrated together to produce a solution of pH 6 citrate buffer.

TABLE 5
Materials for capsaicin formulations both with and without a mucoadhesive.

| | Manufacturer | | Lot # or |
|---|---|---|---|
| Material | or Supplier | Part # | Serial # |
| Capsaicin | Formosa | — | F00112001 |
| Rosemary Extract | Herb Pharm | — | 403118-2F43 |
| Eucalyptol | SAFC | W246506 | MKBL9040V |
| Grapefruit Seed Extract | NutriBiotic | — | 27912-3 |
| Tween 80 | Spectrum | PO138 | XV0879 |
| Glycerol | Spectrum | G1015 | UU0682 |
| Sea Salt | Frontier | — | 2242 |
| Ascorbic Acid | Spectrum | AS102 | WK0144 |
| Citric Acid (•H2O) | Sigma-Aldrich | C1909 | 037K0017 |
| Sodium Citrate | Spectrum | SO165 | YD0763 |
| (•2 H2O) | | | |
| Anhydrous Citric Acid | Spectrum | CI131 | XC0269 |
| Benzalkonium Chloride | Sigma Aldrich | 12063 | BCBH9800V |
| Sinus Buster | SiCap | — | — |
| Sinol-M | Sinol USA | — | — |
| Aloe | Aloe Corp | AA8010XQ-10 | G12066B11 |
| Avicel RC 591 | FMC BioPolymer | RC-591 | DN12824723 |

| | |
|---|---|
| Water | In-house deionized (Barnstead Nanopure) |

TABLE 6
Equipment for capsaicin formulations both with and
without a mucoadhesive.

| Equipment | Description |
|---|---|
| pH meter | Orion 4 Star Plus pH and conductivity meter |
| Balances | Mettler Toledo AB204-S/FACT |
| | Mettler Toledo AX105 |
| | Ohaus CS2000 |
| Osmometer | Precision Systems μOsmette |
| Viscometer | Brookfield LVT |

### Example 5

### Formulation Preparation: Round 1

This example illustrates the preparation of capsaicin formulations without a mucoadhesive for intranasal delivery. Three capsaicin formulations were prepared according to the following procedure on a weight-to-weight basis on a 100 g scale. The compositions of each formulation are summarized in Table 7.

In an appropriate sized beaker, formulation components were weighed, stirred, and sonicated until complete dissolution was achieved in approximately 90% of the total formulation volume. Prior to the addition of the remaining volume of water, the pH and osmolality of each solution was measured and adjusted to obtain isosmotic solutions in the pH range of 6.0-6.5, if possible. Formulations 2 and 3 were adjusted to the appropriate pH range using the 5% citric acid solution that was used to prepare the citrate buffer. Note that the final buffer concentration in Formulations 1 and 2 was 0.5% (w/w) citrate buffer.

The initial 5% (w/w) citrate buffer was diluted 10-fold (w/w) into each formulation. The volumes used for pH adjustment were negligible and were not expected to affect the final buffer concentration. All formulations were observed to be hyperosmotic and thus did not require osmolality adjustments.

Each formulation was then brought to full volume with water and stirred overnight to ensure a homogeneous formulation was obtained and the pH and osmolality were measured. Additionally, the density of each solution was measured in triplicate. These results are summarized in Table 8.

TABLE 7
Composition of capsaicin formulations prepared for Round 1.

| | Formulation (% w/w) | | |
|---|---|---|---|
| Component | 1 | 2 | 3 |
| Capsaicin | 0.027% | 0.027% | 0.0135% |
| Rosemary Extract | 0.08% | — | — |
| Eucalyptol | 0.13% | 0.13% | 0.13% |
| Grapefruit Seed Extract | 0.05% | — | — |
| Tween 80 | 0.50% | 0.50% | 0.50% |
| Glycerol | 3.65% | 3.65% | 3.65% |
| Sea Salt | 0.53% | — | — |
| Ascorbic Acid | 0.83% | — | — |
| Citric Acid | 0.26% | — | — |
| 50 mg/mL citrate buffer, pH 6 | — | 10.0% | 10.0% |
| (5% citrate buffer, pH 6) | | | |
| Purified Water | 93.94% | 85.69% | 85.71% |

TABLE 8
Results of analysis of Round 1 capsaicin formulations.

| | Formulation # | | |
|---|---|---|---|
| Analysis | 1 | 2 | 3 |
| pH | 2.30 | 6.40 | 6.40 |
| Osmolality (mOsm/kg H₂O) | 701 | 523 | 523 |
| Average Density (g/mL)* | 1.015 | 1.014 | 1.015 |

*Density measured at 23.4° C.

### Example 6

### Formulation Preparation: Round 2

A second set of three capsaicin formulations not containing a mucoadhesive were prepared by according to the following protocol on a weight-to-weight basis on a 100 g scale. The compositions of each formulation are summarized in Table 9.

In an appropriate sized beaker, formulation components were weighed, stirred, and sonicated until complete dissolution was achieved in approximately 90% of the total formulation volume. Prior to the addition of the remaining volume of water, the pH and osmolality of each solution was measured and adjusted to obtain isoosmotic solutions at pH 6.0, if possible.

All formulations were adjusted to about pH 6.0 using the 10% citric acid solution that was used to prepare the citrate buffer. Note that the final buffer concentration in all formulations 2.5% (w/w) citrate buffer. The initial 10% (w/w) citrate buffer was diluted 4-fold (w/w) into each formulation.

The volumes used for pH adjustment were negligible and are not expected to affect the final buffer concentration. All formulations were observed to be hyperosmotic and thus did not require osmolality adjustments.

Each formulation was then brought to full weight with water and stirred overnight to ensure a homogeneous formulation was obtained and the pH and osmolality were measured. Additionally, the density of each solution was measured in triplicate. In addition to the prepared formulations, two commercially available nasal sprays were analyzed for pH and osmolality. These results are summarized in Table 10.

TABLE 9
Composition of capsaicin formulations prepared for Round 2.

| | Formulation (% w/w) | | |
|---|---|---|---|
| Component | 1 | 2 | 3 |
| Capsaicin | 0.027% | 0.027% | 0.0135% |
| Rosemary Extract | 0.08% | — | 0.08% |
| Eucalyptol | 0.13% | 0.13% | 0.13% |
| Grapefruit Seed Extract | 0.05% | — | 0.05% |
| Tweets 80 | 0.50% | 0.50% | 0.50% |
| Glycerol | 2.0% | 2.0% | 2.0% |
| 100 mg/mL Citrate, pH 6 | 25.0% | 25.0% | 25.0% |
| (10% citrate buffer, pH 6) | | | |
| Benzalkonium Chloride | 0.10% | 0.10% | 0.10% |
| Purified Water | 72.11% | 72.24% | 72.13% |

TABLE 10
Results of analysis of Round 2 capsaicin formulations and
commercially available nasal sprays.

| | Formulation # | | | Commercial Sprays | |
|---|---|---|---|---|---|
| Analysis | 1 | 2 | 3 | Sinus Buster | Sinol-M |
| pH | 6.05 | 6.04 | 6.04 | 3.48 | 3.72 |
| Osmolality | 641 | 619 | 626 | 797 | 14 |
| (mOsm/kg H₂O) | | | | | |
| Average Density | 1.023 | 1.023 | 1.025 | — | — |
| (g/mL)* | | | | | |

*Density measured at 22.5° C.

### Example 7

### Formulation Preparation: Round 3

This example demonstrates the preparation of capsaicin formulations containing a mucoadhesive for intranasal delivery. A final set of five capsaicin formulations were prepared according to the following procedure on a weight-to-weight basis on a 700 g scale for all formulations except Formulation 2, which was prepared on a 100 g scale. The compositions of each formulation are summarized in Table 11.

To facilitate the preparation of the five formulations, several stock solutions of components were prepared which were then diluted appropriately to target the desired concentrations. The stock solutions are summarized below.

Rosemary Extract (0.8%), Eucalyptol (1.3%), Grapefruit Seed Extract (0.5%), Tween 80 (5%), and Benzalkonium Chloride (0.1%) in water (92.3%)—a 10× stock solution was prepared to contain each of the excipients at 10× greater concentration (w/w) than the desired formulations. This solution was then diluted 10-fold (w/w) into the formulations to obtain the concentrations shown in Table 11.

AVICEL® (2.5%)—a 2× stock solution was prepared to contain AVICEL® at twice the concentration (w/w) than the desired formulations. This solution was prepared in advance of the formulations and allowed overnight stirring to achieve a homogenous dispersion. A cloudy white solution was obtained. This solution was then diluted 2-fold (w/w) to obtain a 1.25% final concentration, as shown in Table 11.

In an appropriate sized beaker, formulation components and/or stock solutions were weighed, stirred, and sonicated until complete dissolution was achieved in approximately 90% of the total formulation volume.

Prior to the addition of the remaining volume of water, the pH and osmolality of each solution was measured and adjusted to obtain isoosmotic solutions at pH 6.0, if possible. All formulations were adjusted to pH 6.0 using the 10% citric acid solution that was used to prepare the citrate buffer. Note that the final buffer concentration in all formulations 0.25% (w/w) citrate buffer. The initial 10% (w/w) citrate buffer was diluted 40-fold (w/w) into each formulation. The volumes used for pH adjustment were negligible and are not expected to affect the final buffer concentration.

All formulations were observed to be nearly isoosmotic and thus did not require osmolality adjustments.

Each formulation was then brought to full weight with water and stirred overnight to ensure a homogeneous formulation was obtained and the pH and osmolality were measured. Additionally, the viscosity of each solution was measured in triplicate. The results are shown in Table 12.

TABLE 11
Composition of capsaicin formulations containing a
mucoadhesive prepared for Round 3.

| | Formulation (% w/w) | | | | |
|---|---|---|---|---|---|
| Component | 1 | 2 | 3 | 4 | 5 |
| Capsaicin | 0.027% | 0.0135% | 0.027% | 0.027% | 0.027% |
| Rosemary Extract | 0.08% | 0.08% | 0.08% | 0.08% | 0.08% |
| Eucalyptol | 0.13% | 0.13% | 0.13% | 0.13% | 0.13% |
| Grapefruit Seed Extract | 0.05% | 0.05% | 0.05% | 0.05% | 0.05% |
| Tween 80 | 0.50% | 0.50% | 0.50% | 0.50% | 0.50% |
| Benzalkonium Chloride | 0.01% | 0.01% | 0.01% | 0.01% | 0.01% |
| Glycerol | 2.0% | 2.0% | 2.0% | 2.0% | 2.0% |
| 100 mg/mL Citrate, pH 6 | 2.5% | 2.5% | 2.5% | 2.5% | 2.5% |
| (10% citrate buffer, pH 6) | | | | | |
| Aloe | 0.01% | 0.01% | 0.01% | — | 0.20% |
| 25 mg/mL Avicel | 50.0% | 50.0% | — | 50.0% | 50.0% |
| (2.5% Avicel) | | | | | |
| Purified Water | 37.76% | 37.78% | 87.76% | 37.77% | 37.57% |

TABLE 12
Results of analysis of Round 3 capsaicin formulations.

| | Formulation # | | | | |
|---|---|---|---|---|---|
| Analysis | 1 | 2 | 3 | 4 | 5 |
| pH | 6.09 | 6.07 | 6.10 | 6.10 | 6.06 |
| Osmolality (mOsm/kg H₂O) | 311 | 314 | 302 | 306 | 356 |
| Average Viscosity (cP) | 10.0 | 12.3 | 1.02 | 8.73 | 8.81 |

### Example 8

### Preservative Studies

This example demonstrates the efficacy of formulations containing capsaicin as a preservative with bactericidal activity. An initial verification screen was performed using phosphate buffer with 0.1% polysorbate 80 (PS80) as diluent, Tryptic Soy Agar (TSA) and Sabouraud Dextrose Agar (SAB). With these testing conditions no recovery of the three bacteria strains was observed, however recovery of the yeast and fungi were acceptable.

USP 51 guideline process was used to assess whether a preservative system containing benzalkonium chloride, capsaicin USP and grapefruit seed extract would be able to withstand a challenge involving the inoculation of microbes and fungi into a growth medium containing the preservative system.

For the purposes of supporting prototype formulation development, a screening verification for appropriate organism recovery and neutralization methodology was performed on a prototype capsaicin, RND114 formulation. USP <51> guidance was used as a basis for performing the verifications.

An initial verification screen was performed using phosphate buffer with 0.1% polysorbate 80 (PS80) as diluent, Tryptic Soy Agar (TSA) and Sabouraud Dextrose Agar (SAB). With these testing conditions no recovery of the three bacteria strains was observed, however recovery of the yeast and fungi were acceptable.

The purpose of this screening verification was to determine if the standard dilution methodology would be effective in recovering organisms when performing a “Preservative Challenge Test” (also known as an Antimicrobial Effectiveness Test, (AET). The prototype organism recovery verification testing was performed by adding the appropriate inoculum of indicator organism suspensions to aliquots of the formulation. Serial dilutions were prepared, starting with a 1:10 dilution of the inoculated test formulations. Subsequently, 1 mL pour plates were prepared from each dilution to evaluate organism recovery efficiency. The five indicator organisms from USP <51> were used. (Incubations could be extended past the standard ranges if inhibition was observed, to collect further information.) USP <51> was referenced as a guide for performing this experimental verification.

Reagents and Materials:

| Reagents/Materials | Lot No. | Exp. Date | Manuf. | |
|---|---|---|---|---|
| Phosphate buffer, pH 7.2, | 277296 0000693874 | Nov. 20, 2013 | Remel | |
| with 0.1% | | | | |
| polysorbate 80 | | Aug. 22, 2013 | Croda | |
| TSA | 258337 | Oct. 10, 2013 | Remel | |
| SAB | 13023 | Jul. 22, 2013 | Hardy | |
| Microrganisms | ATCC | Lot No. | Exp. Date | Manuf. |
| S. aureus | 6538 | 485-146 | March 2014 | Microbiologics |
| P. aeruginosa | 9027 | 484-307 | October 2014 | Microbiologics |
| E. coli | 8739 | 483-315 | April 2015 | Microbiologics |
| C. albicans | 10231 | 443-179 | July 2013 | Microbiologics |
| A. brasiliensis | 16404 | 392-205 | January 2015 | Microbiologics |
| Lot | | ATN # | | |
| RND114 | | SV05-01-062413A001 | | |
| | | SV05-01-053013A001 | | |

### Procedure:

A 10 mL aliquot for each indicator organism (5) was prepared. Commercially purchased organism suspensions to result in a cfu/mL. concentration in the aliquots in the range of 500 cfu/mL (or a total of around 5,000 cfu in the inoculum volume) were also prepared. Theoretical calculations were based on the manufacturer's certificate of analysis.

Each of the 5 indicator organism were inoculated into individual aliquots and mixed well. 1 mL of each inoculated aliquot was transferred into 9 mL of diluent (phosphate buffer, 0.1% PS80) to afford a 1:10 dilution and mixed well. 1 mL of the 1:10 “neutralized” dilution was transferred into duplicate pour plates. (The theoretical cfu count in the pour plates was around 50.) Subsequently, 15-20 mL of the appropriate melted agar for the organism was added, swirled to mix and allowed to solidify. (TSA for bacteria. SAB for yeast/mold.).

The plates were incubated as follows: TSA plates 30-35° C. SAB plates 20-25° C. Standard incubation for bacteria and candida is 3-5 days, therefore expected recovery of the organisms was less than or equal to 3 days. Standard incubation for aspergillus is 5-7 days, therefore expected recovery for this organism was less than or equal to 5 days. (Note: during verification testing, the incubation may have been extended to gather additional information, if needed, for example if inhibition was observed). After incubation, the cfu on each plate was counted and the average of the duplicate plates was calculated. Results were compared to inoculum control counts. Acceptable results were within ±a factor of two of the control count.

An initial verification screen was performed using phosphate buffer with 0.1% polysorbate 80 (PS80) as diluent, Tryptic Soy Agar (TSA) and Sabouraud Dextrose Agar (SAB). Under these testing conditions, no recovery of the three bacteria strains was observed, but the yeast and fungi were found to be acceptable.

Follow up organism recovery studies will be performed to document a successful USP 51 methodology for this formulation. The next steps will be to evaluate several higher neutralizing dilutions using a neutralizing diluent such as TSB with L&T, with standard TSA as the agar. In addition, evaluating the use of the same dilutions, with a neutralizing agar, such as TSA with L&T will be conducted. See the table representations below.

TABLE 13
Follow up Test 1: Use TSA pour plate agar.
Replicate for each organism, SA, PA, EC.

| | 1:10 | 1:20 | 1:50 | 1:100 |
|---|---|---|---|---|
| Volume of | 1 mL | 0.5 mL | 0.2 mL | 0.1 mL |
| 10 mL inoculated | | | | |
| sample | | | | |
| Diluent Volume | 9 mL | 9.5 mL | 9.8 mL | 9.9 mL |
| (TSB w/L&T) | | | | |

TABLE 14
Follow up Test 2: Use TSA with L&T pour plate agar.
Replicate for each organism, SA, PA, EC.

| | 1:10 | 1:20 | 1:50 | 1:100 |
|---|---|---|---|---|
| Volume of | 1 mL | 0.5 mL | 0.2 mL | 0.1 mL |
| 10 mL inoculated | | | | |
| sample | | | | |
| Diluent Volume | 9 mL | 9.5 mL | 9.8 mL | 9.9 mL |
| (TSB w/L&T) | | | | |

### Example 9

### Patient Studies

This example illustrates the efficacy and time to onset in humans of the composition prepared and formulated for delivery by nasal route in Example 7 (i.e., AUSANIL®).

Twelve patients with a history of severe headaches, frequent headaches, migraines or cluster headaches with a positive history of functional disability (inability to work, difficulty concentrating, tiredness, nausea, vomiting, photo and/or phonophobia) during their headaches were treated with the disclosure compositions and formulations of Example 7 (AUSANIL®). Each patient was interviewed by a board certified neurologist (AC), a complete medical history was taken and a physical and neurological exam were completed to ascertain correct diagnosis and appropriateness for AUSANIL® use.

Patients were instructed to take AUSANIL® by nose at the earliest indication of headache onset. AUSANIL® was sprayed in the ipsilateral nose in the case of migraines and cluster headache and bilaterally in the case of bilateral/tension headache. Patients were instructed to not inhale, swallow, or otherwise ingest AUSANIL® and to clear residual medication after approximately one minute by nose-blowing.

Table 15 summarizes the patient series with response and is followed by a brief summary of each patient's history and response. Patient response was based on a five point rating scale, with five being the highest degree of relief.

TABLE 15
Summary of Patient Response to AUSANIL ®.

| Case # | Patient | Diagnosis | Response | Side Effects |
|---|---|---|---|---|
| 1 | 45 year old man | Cluster HA | +++++ | Nasal sting |
| 2 | 32 year old woman | Bi-frontal | +++++ | Nasal sting |
| | | Tension HA | | |
| 3 | 65 year old man | Migraine HA | +++++ | Nasal sting |
| 4 | 46 year old man | Migraine HA | +++++ | Nasal sting |
| 5 | 40 year old woman | Tension HA | ++++ | Nasal sting |
| 6 | 57 year old man | Post traumatic HA | +++ | Prolonged |
| | | | | sting |
| 7 | 36 year old woman | Migraine HA | +++++ | Nasal sting |
| 8 | 29 year old woman | Migraine HA | +++++ | Nasal sting |
| 9 | 49 year old man | Infrequent HA | +++++ | Nasal sting |
| 10 | 44 year old woman | Bi-lateral | +++ | Prolonged |
| | | Tension HA | | sting |
| 11 | 33 year old man | Migraine & | +++++ | Nasal Sting |
| | | Tension HA | | |
| 12 | 28 year old woman | Bilateral HA | ++ | Prolonged |
| | | | | sting |

### Case Summaries:

Case 1: A 45 year old man with cluster headaches for many years suffering from multiple headaches with current cluster lasting a month. Patient suffered 2-3 very severe headaches/day and described wanting to die. Trials of verapamil, indomethacin, prednisone, triptans, and oxygen were unsuccessful. AUSANIL®, used multiple times per day, successfully treated patient's headaches. Patient indicated the nasal sting was of no consequence and that relief was immediate.

Case 2: A 32 Year old woman with daily bifrontal “tension” headaches for several years. Patient history strongly suggests Chronic Frequent headaches related to medication overuse (medication overuse/rebound headaches). Using high dose MOTRIN®/EXCEDRIN®/TYLENOL® (up to 1 g/day) with moderate response to medications and long onset for pain relief. AUSANIL® used bilaterally as a nasal spray successfully treated headaches with immediate onset of action. Patient continued to use AUSANIL® daily with no loss of effectiveness. Nasal sting was described as no consequence to patient. After one month of regular use, patient's headaches have decreased to once or twice a month. AUSANIL® continues to be rapidly effective for patient's headaches.

Case 3: A 65 year old man with a lifelong history of “migraine” headaches. Headaches occur several times a month throughout patient's youth that went away for twenty years and have returned in the last ten years. Headaches occur twice a week but the intensity is much lower than patient's earlier “migraines”. Unilateral throbbing on his temples and using analgesics for pain relief requiring over an hour for analgesics to work. Patient described satisfaction with current prescription. AUSANIL® spray unilaterally on the side of the headache was successful in treating the pain with immediate onset of pain relief and no recurrence for 24 hours. Patient described nasal sting as surprising but not problematic.

Case 4: A 46 year old man with lifelong migraines and headaches since childhood. Headaches occurring unilaterally on the left side several times a month. Taking high dose TYLENOL® (1 g) for headaches. Triptans contraindicated because of history of CAD. Long history of high dose NAPROSYN®/ibuprofen/ASPIRIN® use. Patient diagnosed with gastric ulcer caused by above. Unable to use DHE because of nausea. AUSANIL® use resulted in immediate and sustained relief. Patient has been using AUSANIL® for almost a year with relief of headaches in less than a minute. Nasal sting is not a problem. Patient no longer uses TYLENOL®.

Case 5: A 40 year old woman having occasional tension headaches, some related to menstruation. Headaches occurring two to three times per month for several years. Some headaches occur at the beginning of menstrual period. Multiple OTC medications used to treat headaches including TYLENOL®/NAPROSYN®/ibuprofen. Treatment regimen generally effective, with the exception of menstrual headaches, but have a long onset of action. AUSANIL® use resulted in immediate and complete relief of tension headaches. Patient no takes AUSANIL® with Tylenol for headaches to speed onset of relief. AUSANIL® was ineffective in sustained relief of menstrual headaches: patient described improvement in symptoms, but reported that headache would resume/worsen within approximately 30 minutes.

Case 6: A 57 year old left-handed man with a traumatic brain injury (TBI) resulting chronic severe headaches and poor functioning due to daily pain. Patient fell down a flight of stairs eight years ago and had TBI with subdural hematoma on the left parietal region. Chronic headache syndrome subsequent to injury that was very difficult to manage consisting of constant baseline pain and occasional worsening of pain sharply on the left side. Patient was treated unsuccessfully with Percocet®, oxycodone, Fioricet® and nerve blocks. Only other medical problem is hypercholesterolemia, which was treated with Simvastatin. Patient used AUSANIL® with two puffs twice separated by ten minutes on the left side for pain. Pain relief was experienced in about two minutes and described as a 50-65% improvement and “relaxation” that lasted two and a half hours. Patient reported a surprising sting lasting several minutes, but that sting was of no consequence in light of pain relief benefit.

Case 7: A 36 year old right handed woman with a history of migraines since teens. Headaches occurred at the rate of three or four a month and invariably associated with nausea, vomiting, photo and phonophobia. Headaches impair functioning and preclude patient from working. Patient tried prescription IMITREX®, which did not provide pain relief and caused more nausea. Patient additionally used EXCEDRIN®, MOTRIN®, TYLENOL®, ASPIRIN® and SINOL® with limited and slow response to pain. AUSANIL®, with two sprays administered to ipsilateral nostril resulted in pain relief within 45 seconds. Patient reported sting was of no consequence and no other side effects were observed.

Case 8: A 29 year old right handed woman with migraine headaches since age 7 years diagnosed by a neurologist 4 years ago. Headaches occur mostly on the right side of the head and at a frequency of two to three times per month. Headaches invariably associated with visual auras, tunnel vision, difficulty walking (no ataxia) and severe photo and phono-phobia. Patient experienced nausea and vomiting with each untreated episode. Pain is mostly felt behind right eye and described as a pounding sensation. Patient used IMITREX® for pain relief, but reported too much nausea, and INDERAL®, which caused depression. Patient was using EXCEDRIN® PM, two tablets early at onset of headache followed two more in an hour. Patient reported that headache does not go away in less than an hour with existing treatment regimen. AUSANIL® two sprays to right nostril treated provided patient with relief from migraine pain in less than five minutes. Patient reported sting was of no consequence in view of pain relief. No additional side effects were reported. Subsequent use of AUSANIL® with complete pain relief in less than three minutes each time and no recurrence in 24 hours.

Case 9: A 49 year old right-handed man with occasional severe frontal bilateral headaches described as “bolts of lightning”. No history of any specific headache syndromes, otherwise completely healthy and on no medications for headaches due to the relative infrequency. Patient administered two doses of AUSANIL® to each nostril and reported complete relief of pain from headache in less than two minutes. Patient indicated the sting was of no consequence.

Case 10: A 44 year old right-handed woman with a fifteen year history of bilateral tension headaches triggered by work stress, lack of sleep and occasionally hormone related. Patient experiences three to four headaches each month treated, with recent headaches upon waking that lasted days. Headaches were treated with 160 mg propranolol daily. Patient additionally uses piroxicam, butalbital and hydrocodeine several times for pain. Pain around forehead and temples bilaterally described as pounding with no other symptoms. One dose of AUSANIL® administered to each nostril effectively treated the headache within a couple of minutes. Patient described sting as minor and lasting for approximately ten minutes. Although headache recurred after two hours, subsequent dosing with AUSANIL® was effective in alleviating pain.

Case 11: A 33 year old right-handed man with a history of migraines and tension headaches triggered by work stress, lack of sleep and alcohol. Headaches frequency was two to five times per month. High dose EXCEDRIN® and MOTRIN® were effective, but took over an hour for relief and sometimes multiple doses were required for complete relief. One dose of AUSANIL® to each nostril successfully treated patient's headache in 20 seconds with no recurrence. Patient reported sting was of no consequence in view of pain relief.

Case 12: A 28 year old right-handed woman with a 20 year history of bilateral headaches diagnosed by a neurologist as migraines. Headaches occurred all over the head, accompanied with photophobia and phonophobia. Patient also described back of head and neck pain described variously as sharp and throbbing in parts. Headache invariably accompanied by nausea and vomiting, usually about three hours into the headache. Headaches occur at the rate of once weekly, triggered by exercise, sleep deprivation, red wine, but none related to menstruation. Patient used TOPAMAX®, sumatriptan, herbal medications, EXCEDRIN®, and ADVIL® in the past with variable results and significant functional disability. Patient was experiencing a headache when examined and evaluated by neurologist for AUSANIL® and was treated during visit. Patient reported some relief in a few minutes, however, subsequent headache(s) did not benefit from AUSANIL® use.

In general, the most common side effects of the disclosed compositions were a transient stinging sensation and tearing up or redness of the eyes, and occasionally sneezing. Some cases of mild throat irritation were reported (if the medication drips down the back of the throat).

Although the invention has been described with reference to the above examples, it will be understood that modifications and variations are encompassed within the spirit and scope of the invention. Accordingly, the invention is limited only by the following claims.

What is claimed is:

1. A method for the treatment of a migraine, severe or cluster headache, comprising administering to a subject in need thereof, a therapeutically effective amount of the composition of claim 1, thereby treating the migraine or headache.

2. The method of claim 1, wherein the subject is a human.

3. The method of claim 1, wherein the composition is administered by nasal spray.

4. The method of claim 1, further comprising administering at least one additional therapeutic agent.

5. The method of claim 4, wherein the therapeutic agent is an analgesic.

6. The method of claim 5, wherein the analgesic is a non-steroidal anti-inflammatory (NSAID), triptan, opioid, acetaminophen, a semi-synthetic opioid or dihydroergotamine (DHE).

7. The method of claim 1, further comprising administering oxygen.

8. The method of claim 1, wherein the concentration is about 0.0135% (w/w) capsaicin.

9. The method of claim 1, wherein the composition further comprises a mucoadhesive agent.
